(19) United States
(12) Patent Application Publication
(10) Pub. No.: US 2004/0229855 A1
(43) Pub. Date: Nov. 18, 2004

(54) Antiglucocorticoids for the treatment of postpartum psychosis

(75) Inventor: Joseph K. Belanoff, Woodside, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Corcept Therapeutics, Inc., Menlo Park, CA

(21) Appl. No.: 10/772,919

(22) Filed: Feb. 4, 2004

Related U.S. Application Data
(60) Provisional application No. 60/445,284, filed on Feb. 4, 2003.

(51) Int. Cl. A61K 31/56

(52) U.S. Cl. 514/179

(57) ABSTRACT

This invention generally pertains to the field of psychiatry. In particular, this invention pertains to the discovery that agents which inhibit the binding of cortisol to its receptors can be used in methods for treating postpartum psychosis. Mifepristone, a potent specific glucocorticoid receptor antagonist, can be used in these methods. The invention also provides a kit for treating postpartum psychosis in a human including a glucocorticoid receptor antagonist and instructional material teaching the indications, dosage and schedule of administration of the glucocorticoid receptor antagonist.

### CROSS-REFERENCES TO RELATED APPLICATIONS

This application claims priority to related application U.S. Ser. No. 60/445,284, filed Feb. 4, 2003, which is incorporated herein by reference.

### BACKGROUND OF THE INVENTION

This invention is directed to a method for treating postpartum psychoses. The pathogenesis of postpartum psychosis, which is also known as puerperal psychosis, is related to parturition and childbirth. Postpartum psychosis is an acute mental illness which has a precipitous onset within the first weeks following parturition. Onset of symptoms may occur early, within the first two to four postpartum weeks, or may occur later. Indeed, many clinicians consider symptom onset within six to twelve months after delivery as temporally related to childbirth (Chaudron, L. H. and Pies, R. W. (2003) J. Clin Psychiatry 64(11):1284-1292).

In the general population, postpartum psychosis occurs at a frequency of about 1 or 2 per 1000 births. However, the frequency of occurrence may be higher in certain subpopulations. For example, a recent study found that the frequency of postpartum psychosis among women with bipolar disorder was 260 per 1000 births (Leibenluft, E. (1996) Am. J. Psychiatry 153:163-173).

Postpartum psychosis is characterized by delusions, hallucinations, and an impaired perception of reality. Delusions commonly associated with postpartum psychotic episodes include command hallucinations to kill the infant, delusions that the infant is possessed, or delusions that the infant has been substituted. In at least a subset of women, confusion and disorientation are apparent to a degree not observed in other psychoses.

Patients with postpartum psychosis often present with delusions and hallucinations that are reminiscent of the psychoses of schizophrenia or the various affective disorders that have psychotic features. The symptoms of postpartum psychosis may also sometimes overlap with the symptoms of postpartum blues or depression. However, postpartum psychosis is distinct from the milder forms of depression that occur postpartum, and from other non-puerperal psychoses. Indeed, since most women who develop psychosis following childbirth have no previous history of psychotic illness, there is a strong implication that a distinct syndrome is present.

Studies comparing the symptom presentation of puerperal versus non-puerperal psychosis support the conclusion that puerperal psychosis is a distinct psychotic syndrome. For example, a study comparing 58 women with puerperal psychosis to 52 women with non-puerperal psychosis found that manic symptoms such as elation, mood lability, rambling speech, distractibility, observed euphoria, and increased activity, were significantly more common in the puerperal group. Systematic delusions, persecutory ideas, odd affect, and social withdrawal were more common in the non-puerperal group (Brockington, I. F. et al. (1981) Arch. Gen. Psychiatry 38:829-833).

Postpartum psychosis is a serious illness that may threaten infant well being and which requires hospitalization of the mother. Thus, there is a need for rapid and effective medical treatment of the psychotic symptoms so that the mother infant bond suffers as little damage as possible. This invention supplies that need by providing an effective treatment method for that particular form of psychosis, known as postpartum or puerperal psychosis, which usually has an onset within 4 weeks following childbirth, but which may arise anytime within nine months of parturition.

### BRIEF SUMMARY OF THE INVENTION

The invention is directed to a method of ameliorating the symptoms of postpartum psychosis in a patient in need thereof, comprising administering an amount of a glucocorticoid receptor antagonist effective to ameliorate the symptoms of the postpartum psychosis, with the proviso that the first psychotic symptoms arise within 9 months of childbirth, that the patient has never suffered any psychotic condition not triggered by childbirth, and that the patient did not suffer from psychosis prior to parturition.

In one aspect of the invention, the glucocorticoid receptor antagonist comprises a steroidal skeleton with at least one phenyl-containing moiety in the 11-β position of the steroidal skeleton. In one aspect, the phenyl-containing moiety in the 11-β position of the steroidal skeleton is a dimethylaminophenyl moiety. In another aspect, the glucocorticoid receptor antagonist is mifepristone.

In one aspect of the present invention, the glucocorticoid receptor antagonist is selected from the group consisting of 11β-(4-dimethylaminoethoxyphenyl)-17α-propynyl-17β-hydroxy-4,9 estradien-3-one and 17β-hydroxy-17α-19-(4-methylphenyl)androsta-4,9(11)-dien-3-one. In another aspect, the glucocorticoid receptor antagonist is selected from the group consisting 4α(S)-Benzyl-2(R)-prop-1-ynyl-1,2,3,4,4α,9,10,10α(R)-octahydro-phenanthrene-2,7-diol and 4α(S)-Benzyl-2(R)-chloroethynyl-1,2,3,4,4α,9,10,10α(R)-octahydro-phenanthrene-2,7-diol.

In one aspect, the glucocorticoid receptor antagonist is (11β,17β)-11-(1,3-benzodioxol-5-yl)-17-hydroxy-17-(1-propynyl)estra-4,9-dien-3-one.

In one aspect of the present invention, the administration is once per day. In another aspect, the mode of administration is oral. In yet another aspect, the mode of administration is by a transdermal application, by a nebulized suspension, or by an aerosol spray

The present invention also provides a kit for treating postpartum psychosis in a subject. The kit comprises a specific glucocorticoid receptor antagonist and an instructional material teaching the indications, dosage and schedule of administration of the glucocorticoid receptor antagonist to a patient suffering from postpartum psychosis.

### DEFINITIONS

The term “psychotic” as used herein refers to a psychiatric condition in its broadest sense, as defined in the DSM-IV-TR (American Psychiatric Association: Diagnostic and Statistical Manual of Mental Disorders, Fourth Edition, Text Revision, Washington D.C., American Psychiatric Association (2000)) and as described below. The term “psychotic” has historically received a number of different definitions, ranging from narrow to broadly described. A psychotic condition can include delusions or prominent hallucinations, including prominent hallucinations that the individual realizes are hallucinatory experiences, and those with hallucinations occurring in the absence of insight into their pathological nature. Finally, the term includes a psychotic condition characterized by a loss of ego boundaries or a gross impairment in reality testing. Unlike this definition, which is broad and based primarily on symptoms, the characterization of psychosis in earlier classifications (e.g., DSM-II and ICD-9) was more inclusive and focused on the severity of functional impairment (so that a mental disorder was termed “psychotic” if it resulted in “impairment” that grossly interferes with the capacity to meet ordinary demands of life). Different disorders which have a psychotic component comprise different aspects of this definition of “psychotic.” For example, in schizophreniform disorder, schizoaffective disorder and brief psychotic disorder, the term “psychotic” refers to delusions, any prominent hallucinations, disorganized speech, or disorganized or catatonic behavior. In psychotic disorder due to a general medical condition and in substance-induced psychotic disorder, “psychotic” refers to delusions or only those hallucinations that are not accompanied by insight. Finally, in delusional disorder and shared psychotic disorder, “psychotic” is equivalent to “delusional” (see DSM-IV-TR, supra, page 327-328).

Objective tests can be used to determine whether an individual is psychotic and to measure and assess the success of a particular treatment schedule or regimen. For example, measuring changes in cognitive ability aids in the diagnosis and treatment assessment of the psychotic patient. Any test known in the art can be used, such as the so-called “Wallach Test,” which assesses recognition memory (see below, Wallach (1980) J. Gerontol. 35:371-375), or the Stroop Color and Word Test (“Stroop Test”) (see Golden, C. J., Cat. No. 30150M, In: A Manual for Clinical and Experimental Uses, Stoelting, Wood Dale, Ill.).

The term “psychosis” refers to a psychiatric symptom, condition or syndrome in its broadest sense, as defined in the DSM-IV-TR (2000, supra), comprising a “psychotic” component, as broadly defined above. The term psychosis can refer to a symptom associated with a general medical condition, a disease state or other condition, such as a side effect of drug abuse (a substance-induced disorder) or as a side effect of a medication. Alternatively, the term psychosis can refer to a condition or syndrome not associated with any disease state, medical condition, drug intake or the like.

Psychosis is typically defined as a mental disorder or condition causing gross distortion or disorganization of a person's mental capacity, affective response, or capacity to recognize reality, communicate, and relate to others to the degree of interfering with her capacity to cope with the ordinary demands of everyday life.

The term “postpartum or puerperal psychosis” refers to an acute mental illness which has a sudden onset within the early months following parturition. In many cases symptom onset occurs within the first four weeks postpartum, sometimes within two weeks postpartum, eight weeks postpartum, 12 weeks postpartum or 16 weeks postpartum. However, symptom onset within nine months after delivery is considered to be temporally related to childbirth, and thus, is defined as postpartum psychosis. The illness is characterized by delusions, hallucinations, and an impaired perception of reality.

Delusions commonly associated with postpartum psychotic episodes include command hallucinations to kill the infant, delusions that the infant is possessed, or delusions that the infant has been substituted. In at least a subset of women, confusion and disorientation are apparent to a degree not observed in other psychoses. Postpartum psychoses occur in about 1 in 500 to about 1 in 1000 deliveries, and may be more common in primiparous women. Postpartum psychosis is distinct from the milder forms of depression that occur postpartum. For example, postpartum blues and postpartum depression typically present with depressive features of differing severity. In contrast, postpartum psychosis may present in any mood state including depressed, manic, or mixed, and is the only postpartum syndrome that presents with delusions and hallucinations. Furthermore, the postpartum psychosis patient has no prior history of psychotic disorders unless they were also childbirth associated disorders.

The phrase “the patient has never suffered any psychotic condition not triggered by childbirth, and that the patient did not suffer from psychosis prior to parturition” means that a patient is not suffering from any psychiatric condition known in the art to share symptomatic features with postpartum psychosis, but which is not triggered by childbirth, and that the patient did not suffer from any psychotic condition at any time prior to childbirth, unless that psychosis was also a childbirth associated illness.

Psychiatric conditions known in the art to share symptomatic features with postpartum psychosis include Brief Psychotic Disorder, Psychotic Disorder Due to a General Medical Condition, Substance-Induced Psychotic Disorder, and Schizophrenia (DSM-IV, 2000, page 343) as well as are Mood Disorders with Psychotic Features such as the psychoses associated with Major depression, and Manic or Mixed episode depression.

The term “parturition” refers to childbirth and labor.

The term “ameliorating” or “ameliorate” refers to any indicia of success in the treatment of a pathology or condition, including any objective or subjective parameter such as abatement, remission or diminishing of symptoms or an improvement in a patient's physical or mental well-being. Amelioration of symptoms can be based on objective or subjective parameters; including the results of a physical examination and/or a psychiatric evaluation. For example, a clinical guide to monitor the effective amelioration of a psychiatric disorder, such as psychosis or depression, is found in the Structured Clinical Interview for DSM-IV Axis I mood disorders (“SCID-I/P”), which is a semi-structured diagnostic interview designed to assist clinicians, researchers, and trainees in making the major DSM-IV Axis I diagnoses (see, e.g. SCID-I/P (for DSM-IV-TR) Patient Edition First, Michael B., Spitzer, Robert L, Gibbon Miriam, and Williams, Janet B. W.: Structured Clinical Interview for DSM-IV-TR Axis I Disorders, Research Version, Patient Edition. (SCID-I/P) New York: Biometrics Research, New York State Psychiatric Institute, 2001, DSM-IV-TR, (2000) supra, and Comprehensive Textbook of Psychiatry/VI, vol. 1, sixth ed., pp 621-627, Williams & Wilkins, Balt., Md.).

The term “cortisol” refers to a family of compositions also referred to as hydrocortisone, and any synthetic or natural analogues thereof.

The term “glucocorticoid receptor” refers to a family of intracellular receptors also referred to as the cortisol receptor, which specifically bind to cortisol and/or cortisol analogs. The term includes isoforms of glucocorticoid receptors, recombinant glucocorticoid receptors and mutated glucocorticoid receptors.

The term “mifepristone” refers to a family of compositions also referred to as RU486, or RU38.486, or 17-β-hydroxy-11-β-(4-dimethyl-aminophenyl)-17-α-(1-propynyl)-estra-4,9-dien-3-one), or 11-β-(4dimethylaminophenyl)-17-β-hydroxy-17-α-(1-propynyl)-estra-4,9-dien-3-one), or analogs thereof, which bind to the glucocorticoid receptor, typically with high affinity, and inhibit the biological effects initiated/ mediated by the binding of any cortisol or cortisol analogue to a glucocorticoid receptor. Chemical names for RU-486 vary; for example, RU486 has also been termed: 11β-[p-(Dimethylamino)phenyl]-17β-hydroxy-17-(1-propynyl)-estra-4,9-dien-3-one; 11β-(4-dimethyl-aminophenyl)-17β-hydroxy-17α-(prop-1-ynyl)-estra-4,9-dien-3-one; 17β-hydroxy-11β-(4-dimethylaminophenyl-1)-17α-(propynyl-1)-estra-4,9-diene-3-one; 17β-hydroxy-11β-(4-dimethylaminophenyl-1)-17β-(propynyl-1)-ε; (11b, 17β)-11-[4-dimethylamino)-phenyl]-17-hydroxy-17-(1-propynyl)estra-4,9-dien-3-one; and 11β-[4-(N,N-dimethylamino) phenyl]-17α-(prop-1-ynyl)-D-4,9-estradiene-17β-ol-3-one.

The term “specific glucocorticoid receptor antagonist” refers to any composition or compound which partially or completely inhibits (antagonizes) the binding of a glucocorticoid receptor agonist, such as cortisol, or cortisol analogs, synthetic or natural, to a glucocorticoid receptor. A “specific glucocorticoid receptor antagonist” also refers to any composition or compound which inhibits any biological response associated with the binding of a glucocorticoid receptor to an agonist. By “specific”, we intend the drug to preferentially bind to the glucocorticoid receptor rather than the mineralocorticoid receptor with an affinity at least 100-fold, and frequently 1000-fold.

### DETAILED DESCRIPTION OF THE INVENTION

Treating Postpartum Psychosis in a Subject Using Glucocorticoid Receptor Antagonists

Antiglucocorticoids, such as mifepristone, are formulated as pharmaceuticals to be used in the methods of the invention to treat postpartum psychosis in a subject. Any composition or compound that can block a biological response associated with the binding of cortisol or a cortisol analogue to a glucocorticoid receptor can be used as a pharmaceutical in the invention. Routine means to determine glucocorticoid receptor antagonist drug regimens and formulations to practice the methods of the invention are well described in the patent and scientific literature, and some illustrative examples are set forth below.

Diagnosing and Assessing conditions and Illnesses Involving Psychosis

There are numerous means to diagnose postpartum psychosis and assess the success of treatment. These means include classical psychological evaluations in addition to the various laboratory procedures. Such means are well-described in the scientific and patent literature, and some illustrative examples are provided below.

A. Assessing and Diagnosing Psychosis

The psychosis ameliorated in the methods of the invention encompasses a broad range of mental conditions and symptoms, but all share the common feature that they appear precipitously within the first nine months following parturition. While the practitioner can use any set of prescribed or empirical criteria to diagnose the presence of a postpartum psychosis as an indication to practice the methods of the invention, some illustrative diagnostic guidelines and examples of relevant symptoms and conditions are described below.

Psychosis can be diagnosed by formal psychiatric assessment using, for example, a semi-structured clinical interview described as “The Structured Clinical Interview for DSM-IV, or “SCID.” SCID is designed to be administered by clinicians and researchers familiar with the diagnostic criteria used in the DSM-IV. The SCID has two parts, one for Axis I disorders (clinical disorders and other conditions that may be a focus of clinical attention, including schizophrenia and other psychotic disorders, as well as mood and anxiety disorders) and another for Axis II personality disorders (personality disorders and mental retardation, see DSM-IV-TR, supra, pgs 27-37, for a general description of a “multiaxial assessment system” to guide clinicians in planning treatment and predicting outcome). At the start of the SCID interview, an overview of the present illness, chief complaint, and past episodes of major psychopathology are obtained before systematically asking the patient questions about specific symptoms. The interview schedule itself has many questions which are open ended so that patients have an opportunity to describe symptoms in their own words.

At the conclusion of the interview, the interviewer also completes the Global Assessment of Functioning (GAF) scale, the fifth (“V”) Axis on DSM-IV's multiaxial assessment system. Axis V is for reporting the clinician's judgment of the individual's overall level of functioning. This information is useful in planning treatment and measuring its impact, and in predicting outcome. The GAF scale is particularly useful in tracking the clinical progress of individuals in global terms using a single measure (see DSM-IV-TR, supra, page 34, supra). In some settings, it may be useful to assess social and occupational disability and to track progress in rehabilitation independent of the severity of the psychological symptoms. For this purpose, use, for example, the proposed Social and Occupational Functioning Assessment Scale (SOFAS) DSM-IV-TR, supra, pg. 817-818, Appendix B. Additional assessment schemes can be used, for example, the Global Assessment of Relational Functioning (GARF) Scale (DSM-IV-TR, supra, pg 814-816, Appendix B) or the Defensive Functioning Scale (DSM-IV-TR, supra, pg 807-813, Appendix B).

To assess the progress of a treatment for psychosis or aid in its diagnosis or prognosis, the “Brief Psychiatric Rating Scale (BPRS)” can also be used after the semistructured interview with the patient. The BPRS is an 18-dimension rating scale. Each dimension represents a domain of behavior and psychiatric symptoms, such as anxiety, hostility, affect, guilt and orientation. These are rated on a seven-point “Likert Scale” from “not present” to “extremely severe.” The BPRS is brief, easily learned and provides a quantitative score that reflects global pathology. The BPRS is useful in providing a crude barometer of a patient's overall benefit from treatment, and thus is useful in assessing changes in an individual's condition after treatment and amelioration using the methods of the invention (Overall, J. E. and Gorham, D. R. (1962) Psychol. Reports 10:799).

Objective tests can be also be used with these subjective, diagnostic criteria to determine whether an individual is psychotic and to measure and assess the success of a particular treatment schedule or regimen. Diagnosis, categorization, or assessment of treatment of psychosis or any psychiatric condition can be objectively assessed using any test known in the art, such as that described by Wallach (1980) J. Gerontol. 35:371-375, or the Stroop Color and Word Test.

The so-called “Wallach Test” can measure the presence and degree of psychosis by evaluating cognitive changes in the individual, and assessing recognition memory.

The Stroop Color and Word Test (“Stroop Test”) is another means to objectively determine whether an individual is psychotic and to measure efficacy of treatment (see Golden, supra). The Stroop Test can differentiate between individuals with psychosis and those without. Briefly, the test developed from the observation that the naming of colors is always slower than the reading of color names in literate adults. For instance, it always takes less time to read the printed word “yellow” than it does to recognize what color a word is printed in (for example, “XXX” printed in yellow ink). Furthermore, if color words are printed in non-matching colored inks (as, the word yellow in red ink), it takes a normal individual 50% longer to name the proper color (red) than if they are shown only the color (such as a red rectangle, or “XXX” in red). This delay in color recognition is called “the color-word interference effect” and is the time variable parameter measured in the Stroop Test. The greater the delay, the lower the Stroop Test score (see also Uttl (1997) J. Clin. Exp. Neuropsychol. 19:405-420). Individuals with psychosis have significantly lower scores on the Stroop Test than individuals without psychosis.

Psychiatric conditions, such as postpartum psychosis, can be further diagnosed and evaluated using any of the many tests or criteria well-known and accepted in the fields of psychology or psychiatry.

The features (symptoms) of and criteria for diagnosing psychotic disorders, such as postpartum psychosis, are further described DSM-IV-TR, supra. The DSM-IV-TR classifies postpartum psychosis as condition or illness involving psychosis which cannot be classified as any other psychotic disorder. The affliction includes psychotic symptomology (i.e. delusions, hallucinations, disorganized speech, grossly disorganized or catatonic behavior) that do not meet the criteria for any specific psychotic disorder.

While the practitioner can use any criteria or means to evaluate whether an individual is psychotic to practice the methods of the invention, the DSM-IV-TR sets forth a generally accepted standard for such diagnosing, categorizing and treating of psychiatric disorders, including psychosis. Several illustrative examples of such criteria utilized in the methods of the invention are set forth below.

Psychosis generally is characterized as a mental disorder or condition causing gross distortion or disorganization of a person's mental capacity, affective response, and capacity to recognize reality, communicate, and relate to others to the degree of interfering with his capacity to cope with the ordinary demands of everyday life. Often, delusions or hallucinations are present.

In postpartum psychosis, the content of the delusions or hallucinations may have infanticidal themes including command hallucinations to kill the infant or delusions that the infant is possessed. Postpartum psychosis can also include delusions or hallucinations that have a manic theme. For example, the mother may have delusions that God's voice can be heard explaining the baby is a messiah, or alternatively, the delusions may be persecutory delusions. Further, postpartum psychosis may include symptoms comprising disorganized speech (e.g. frequent derailment or incoherence) and grossly disorganized or catatonic behavior. Clearly, the presence of such delusional thoughts about the infant is associated with significantly increased of harm to the infant.

A diagnosis of postpartum psychosis requires that the psychotic symptoms described above appear within the first nine months following parturition, and that the woman had no prior history of psychotic episodes unless they were also childbirth related.

Treatment of Conditions and Illnesses Associated with Psychosis Using Glucocorticoid Receptor Antagonists

A. Steroidal Anti-Glucocorticoids as Glucocorticoid Receptor Antagonists.

Steroidal glucocorticoid antagonists are administered to treat postpartum psychosis in various embodiments of the invention. Steroidal antiglucocorticoids can be obtained by modification of the basic structure of glucocorticoid agonists, i.e., varied forms of the steroid backbone. The structure of cortisol can be modified in a variety of ways. The two most commonly known classes of structural modifications of the cortisol steroid backbone to create glucocorticoid antagonists include modifications of the 11-β hydroxy group and modification of the 17-β side chain (see, e.g., Lefebvre, J. Steroid Biochem. 33:557-563, 1989).

Examples of steroidal glucocorticoid receptor antagonists include androgen-type steroid compounds as described in U.S. Pat. No. 5,929,058, and the compounds disclosed in U.S. Pat. Nos. 4,296,206; 4,386,085; 4,447,424; 4,477,445; 4,519,946; 4,540,686; 4,547,493; 4,634,695; 4,634,696; 4,753,932; 4,774,236; 4,808,710; 4,814,327; 4,829,060; 4,861,763; 4,912,097; 4,921,638; 4,943,566; 4,954,490; 4,978,657; 5,006,518; 5,043,332; 5,064,822; 5,073,548; 5,089,488; 5,089,635; 5,093,507; 5,095,010; 5,095,129; 5,132,299; 5,166,146; 5,166,199; 5,173,405; 5,276,023; 5,380,839; 5,348,729; 5,426,102; 5,439,913; 5,616,458, 5,696,127 and 6,303,591. Such steroidal glucocorticoid receptor antagonists include cortexolone, dexamethasone-oxetanone, 19-nordeoxycorticosterone, 19-norprogesterone, cortisol-21-mesylate; dexamethasone-21-mesylate, 11β-(4-dimethylaminoethoxyphenyl)-17α-propynyl-17β-hydroxy-4,9 estradien-3-one (RU009), and 17β-hydroxy-17α-19-(4-methylphenyl)androsta-4,9(11)-dien-3-one (RU044).

Other examples of steroidal antiglucocorticoids are disclosed in Van Kampen et al. (2002) Eur. J. Pharmacol. 457(2-3):207, WO 03/043640, EP 0 683 172 B1, and EP 0 763 541 B1, each of which is incorporated herein by reference. EP 0 763 541 B1 and Hoyberg et al., Int'l J. of Neuro-psychopharmacology, 5: Supp. 1, S148 (2002); disclose the compound (11β,17β)-11-(1,3-benzodioxol-5-yl)-17-hydroxy-17-(1-propynyl)estra-4,9-dien-3-one (ORG 34517) which in one embodiment, is administered in an amount effective to ameliorate the symptoms of postpartum psychosis in a patient in need thereof.

1. Removal or Substitution of the 11-β Hydroxy Group

Glucocorticoid agonists with modified steroidal backbones comprising removal or substitution of the 11-β hydroxy group are administered in one embodiment of the invention. This class includes natural antiglucocorticoids, including cortexolone, progesterone and testosterone derivatives, and synthetic compositions, such as mifepristone (Lefebvre, et al. supra). Preferred embodiments of the invention include all 11-β-aryl steroid backbone derivatives because these compounds are devoid of progesterone receptor (PR) binding activity (Agarwal, FEBS 217:221-226, 1987). Another preferred embodiment comprises an 11-β phenyl-aminodimethyl steroid backbone derivative, i.e., mifepristone, which is both an effective anti-glucocorticoid and anti-progesterone agent. These compositions act as reversibly-binding steroid receptor antagonists. For example, when bound to a 11-β phenyl-aminodimethyl steroid, the steroid receptor is maintained in a conformation that cannot bind its natural ligand, such as cortisol in the case of glucocorticoid receptor (Cadepond, 1997, supra).

Synthetic 11-β phenyl-aminodimethyl steroids include mifepristone, also known as RU486, or 17-β-hydrox-11-β-(4-dimethyl-aminophenyl)17-α-(1-propynyl)estra-4,9-dien-3-one). Mifepristone has been shown to be a powerful antagonist of both the progesterone and glucocorticoid (glucocorticoid receptor) receptors. Another 11-β phenyl-aminodimethyl steroids shown to have glucocorticoid receptor antagonist effects includes RU009 (RU39.009), 11-β-(4-dimethyl-aminoethoxyphenyl)-17-α-(propynyl-17 β-hydroxy-4,9-estradien-3-one) (see Bocquel, J. Steroid Biochem. Molec. Biol. 45:205-215, 1993). Another glucocorticoid receptor antagonist related to RU486 is RU044 (RU43.044) 17-β-hydrox-17-α-19-(4-methyl-phenyl)-androsta-4,9 (11)-dien-3-one) (Bocquel, 1993, supra). See also Teutsch, Steroids 38:651-665, 1981; U.S. Pat. Nos. 4,386,085 and 4,912,097.

One embodiment includes compositions containing the basic glucocorticoid steroid structure which are irreversible anti-glucocorticoids. Such compounds include α-keto-methanesulfonate derivatives of cortisol, including cortisol-21-mesylate (4-pregnene-11-β, 17-α, 21-triol-3,20-dione-21-methane-sulfonate and dexamethasone-21-mesylate (16-methyl-9 α-fluoro-1,4-pregnadiene-11β, 17-α, 21-triol-3, 20-dione-21-methane-sulfonate). See Simons, J. Steroid Biochem. 24:25-32, 1986; Mercier, J. Steroid Biochem. 25:11-20, 1986; U.S. Pat. No. 4,296,206.

2. Modification of the 17-β Side Chain Group

Steroidal antiglucocorticoids which can be obtained by various structural modifications of the 17-β side chain are also used in the methods of the invention. This class includes synthetic antiglucocorticoids such as dexamethasone-oxetanone, various 17, 21-acetonide derivatives and 17-β-carboxamide derivatives of dexamethasone (Lefebvre, 1989, supra; Rousseau, Nature 279:158-160, 1979).

3. Other Steroid Backbone Modifications

glucocorticoid receptor antagonists used in the various embodiments of the invention include any steroid backbone modification which effects a biological response resulting from a glucocorticoid receptor-agonist interaction. Steroid backbone antagonists can be any natural or synthetic variation of cortisol, such as adrenal steroids missing the C-19 methyl group, such as 19-nordeoxycorticosterone and 19-norprogesterone (Wynne, Endocrinology 107:1278-1280, 1980).

In general, the 11-β side chain substituent, and particularly the size of that substituent, can play a key role in determining the extent of a steroid's antiglucocorticoid activity. Substitutions in the A ring of the steroid backbone can also be important. 17-hydroxypropenyl side chains generally decrease antiglucocorticoid activity in comparison to 17-propinyl side chain containing compounds.

Additional glucocorticoid receptor antagonists known in the art and suitable for practice of the invention include 21-hydroxy-6,19-oxidoprogesterone (see Vicent, Mol. Pharm. 52:749-753, 1997), Org31710 (see Mizutani, J Steroid Biochem Mol Biol 42(7):695-704, 1992), RU43044, RU40555 (see Kim, J Steroid Biochem Mol Biol. 67(3):213-22, 1998), RU28362, and ZK98299.

B. Non-Steroidal Anti-Glucocorticoids as Antagonists.

Non-steroidal glucocorticoid antagonists are also used in the methods of the invention to treat postpartum psychosis in a subject. These include synthetic mimetics and analogs of proteins, including partially peptidic, pseudopeptidic and non-peptidic molecular entities. For example, oligomeric peptidomimetics useful in the invention include (α-β-unsaturated) peptidosulfonamides, N-substituted glycine derivatives, oligo carbamates, oligo urea peptidomimetics, hydrazinopeptides, oligosulfones and the like (see, e.g., Amour, Int. J. Pept. Protein Res. 43:297-304, 1994; de Bont, Bioorganic & Medicinal Chem. 4:667-672, 1996). The creation and simultaneous screening of large libraries of synthetic molecules can be carried out using well-known techniques in combinatorial chemistry, for example, see van Breemen, Anal Chem 69:2159-2164, 1997; and Lam, Anticancer Drug Des 12:145-167, 1997. Design of peptidomimetics specific for glucocorticoid receptor can be designed using computer programs in conjunction with combinatorial chemistry (combinatorial library) screening approaches (Murray, J. of Computer-Aided Molec. Design 9:381-395, 1995; Bohm, J. of Computer-Aided Molec. Design 10:265-272, 1996). Such “rational drug design” can help develop peptide isomerics and conformers including cycloisomers, retro-inverso isomers, retro isomers and the like (as discussed in Chorev, TibTech 13:438-445, 1995).

Examples of non-steroidal glucocorticoid receptor antagonists include clotrimazole; N (triphenylmethyl)imidazole; N-([2-fluoro-9-phenyl]fluorenyl)imidazole; N-([2-pyridyl]diphenylmethyl)imidazole; N (2 [4,4′,4″-trichlorotrityl]oxyethyl)morpholine; 1-(2 [4,4′,4″-trichlorotrityl]oxyethyl)-4 (2 hydroxyethyl)piperazine dimaleate; N-([4,4′,4″]-trichlorotrityl)imidazole; 9-(3-mercapto-1,2,4 triazolyl)-9-phenyl-2,7-difluorofluorenone; 1-(2-chlorotrityl)-3,5-dimethylpyrazole; 4 (morpholinomethyl)-A-(2-pyridyl)benzhydrol; 5-(5-methoxy-2-(N-methylcarbamoyl)-phenyl)dibenzosuberol; N-(2-chlorotrityl)-L-prolinol acetate; 1-(2-chlorotrityl)-2-methylimidazole; 1 (2 chlorotrityl)-1,2,4-triazole; 1,S-bis(4,4′,4″-trichlorotrityl)-1,2,4-triazole-3-thiol; and N ((2,6 dichloro-3 methylphenyl)diphenyl)methylimidazole (see U.S. Pat. No. 6,051,573); the glucocorticoid receptor antagonist compounds disclosed in U.S. Pat. No. 5,696,127 and 6,570,020; the GR antagonist compounds disclosed in US Patent Application 20020077356, the glucocorticoid receptor antagonists disclosed in Bradley et al., J. Med. Chem. 45, 2417-2424 (2002), e.g., 4α(S)-Benzyl-2(R)-chloroethynyl-1,2,3,4,4α,9,10,10α(R)-octahydro-phenanthrene-2,7-diol (“CP 394531”) and 4α(S)-Benzyl-2(R)-prop-1-ynyl-1,2,3,4,4α,9,10,10α(R)-octahydro-phenanthrene-2,7-diol (“CP 409069”); the compound (11β,17β)-11-(1,3-benzodioxol-5-yl)-17-hydroxy-17-(1-propynyl)estra-4,9-dien-3-one (“ORG 34517”) disclosed in Hoyberg et al., Int'l J. of Neuro-psychopharmacology, 5:Supp. 1, S148 (2002); the compounds disclosed in PCT International Application No. WO 96/19458, which describes non-steroidal compounds which are high-affinity, highly selective antagonists for steroid receptors, such as 6-substituted-1,2-dihydro-N protected-quinolines; and some κ opioid ligands, such as the κ opioid compounds dynorphin-1,13 diamide, U50,488 (trans-(1R,2R)-3,4-dichloro-N-methyl-N-[2-(1-pyrrolidinyl)cyclohexyl]benzeneacetamide), bremazocine and ethylketocyclazocine; and the non-specific opioid receptor ligand, naloxone, as disclosed in Evans et al., Endocrin., 141:2294 2300 (2000).

C. Identifying Specific Glucocorticoid Receptor Antagonists

Because any specific glucocorticoid receptor antagonist can be used to treat postpartum psychosis in a subject, in addition to the compounds and compositions described above, additional useful glucocorticoid receptor antagonists can be determined by the skilled artisan. A variety of such routine, well-known methods can be used and are described in the scientific and patent literature. They include in vitro and in vivo assays for the identification of additional glucocorticoid receptor antagonists. A few illustrative examples are described below.

One assay that can be used to identify a glucocorticoid receptor antagonist of the invention measures the effect of a putative glucocorticoid receptor antagonist on tyrosine amino-transferase activity in accordance with the method of Granner, Meth. Enzymol. 15:633, 1970. This analysis is based on measurement of the activity of the liver enzyme tyrosine amino-transferase (TAT) in cultures of rat hepatoma cells (RHC). TAT catalyzes the first step in the metabolism of tyrosine and is induced by glucocorticoids (cortisol) both in liver and hepatoma cells. This activity is easily measured in cell extracts. TAT converts the amino group of tyrosine to 2-oxoglutaric acid. P-hydroxyphenylpyruvate is also formed. It can be converted to the more stable p-hydroxybenzaldehyde in an alkaline solution and quantitated by absorbance at 331 nm. The putative glucocorticoid receptor antagonist is co-administered with cortisol to whole liver, in vivo or ex vivo, or hepatoma cells or cell extracts. A compound is identified as a glucocorticoid receptor antagonist when its administration decreases the amount of induced TAT activity, as compared to control (i.e., only cortisol or glucocorticoid receptor agonist added) (see also Shirwany, Biochem. Biophys. Acta 886:162-168, 1986).

Further illustrative of the many assays which can be used to identify compositions utilized in the methods of the invention, in addition to the TAT assay, are assays based on glucocorticoid activities in vivo. For example, assays that assess the ability of a putative glucocorticoid receptor antagonist to inhibit uptake of ³H-thymidine into DNA in cells which are stimulated by glucocorticoids can be used. Alternatively, the putative glucocorticoid receptor antagonist can complete with ³H -dexamethasone for binding to a hepatoma tissue culture glucocorticoid receptor (see, e.g., Choi, et al., Steroids 57:313-318, 1992). As another example, the ability of a putative glucocorticoid receptor antagonist to block nuclear binding of ³H -dexamethasone-glucocorticoid receptor complex can be used (Alexandrova et al., J. Steroid Biochem. Mol. Biol. 41:723-725, 1992). To further identify putative glucocorticoid receptor antagonists, kinetic assays able to discriminate between glucocorticoid agonists and antagonists by means of receptor-binding kinetics can also be used (as described in Jones, Biochem J. 204:721-729, 1982).

In another illustrative example, the assay described by Daune, Molec. Pharm. 13:948-955, 1977; and in U.S. Pat. No. 4,386,085, can be used to identify anti-glucocorticoid activity. Briefly, the thymocytes of adrenalectomized rats are incubated in nutritive medium containing dexamethasone with the test compound (the putative glucocorticoid receptor antagonist) at varying concentrations. ³H -uridine is added to the cell culture, which is further incubated, and the extent of incorporation of radiolabel into polynucleotide is measured. Glucocorticoid agonists decrease the amount of 3H-uridine incorporated. Thus, a glucocorticoid receptor antagonist will oppose this effect.

For additional compounds that can be utilized in the methods of the invention and methods of identifying and making such compounds, see U.S. Pat. Nos. 4,296,206 (see above); 4,386,085 (see above); 4,447,424; 4,477,445; 4,519,946; 4,540,686; 4,547,493; 4,634,695; 4,634,696; 4,753,932; 4,774,236; 4,808,710; 4,814,327; 4,829,060; 4,861,763; 4,912,097; 4,921,638; 4,943,566; 4,954,490; 4,978,657; 5,006,518; 5,043,332; 5,064,822; 5,073,548; 5,089,488; 5,089,635; 5,093,507; 5,095,010; 5,095,129; 5,132,299; 5,166,146; 5,166,199; 5,173,405; 5,276,023; 5,380,839; 5,348,729; 5,426,102; 5,439,913; and 5,616,458; and WO 96/19458, which describes non-steroidal compounds which are high-affinity, highly selective modulators (antagonists) for steroid receptors, such as 6-substituted-1,2-dihydro N-1 protected quinolines.

The specificity of the antagonist for the glucocorticoid receptor relative to the MR can be measured using a variety of assays known to those of skill in the art. For example, specific antagonists can be identified by measuring the ability of the antagonist to bind to the glucocorticoid receptor compared to the MR (see, e.g., U.S. Pat. Nos. 5,606,021; 5,696,127; 5,215,916; 5,071,773). Such an analysis can be performed using either direct binding assay or by assessing competitive binding to the purified glucocorticoid receptor or MR in the presence of a known antagonist. In an exemplary assay, cells that are stably expressing the glucocorticoid receptor or mineralocorticoid receptor (see, e.g., U.S. Pat. No. 5,606,021) at high levels are used as a source of purified receptor. The affinity of the antagonist for the receptor is then directly measured. Those antagonists that exhibit at least a 100-fold higher affinity, often 1000-fold, for the glucocorticoid receptor relative to the MR are then selected for use in the methods of the invention.

A glucocorticoid receptor-specific antagonist may also be defined as a compound that has the ability to inhibit glucocorticoid receptor-mediated activities, but not MR-mediated activities. One method of identifying such a glucocorticoid receptor-specific antagonist is to assess the ability of an antagonist to prevent activation of reporter constructs using transfection assays (see, e.g., Bocquel et al, J. Steroid Biochem Molec. Biol. 45:205-215, 1993; U.S. Pat. Nos. 5,606,021, 5,929,058). In an exemplary transfection assay, an expression plasmid encoding the receptor and a reporter plasmid containing a reporter gene linked to receptor-specific regulatory elements are cotransfected into suitable receptor-negative host cells. The transfected host cells are then cultured in the presence and absence of a hormone, such as cortisol or analog thereof, able to activate the hormone responsive promoter/enhancer element of the reporter plasmid. Next the transfected and cultured host cells are monitored for induction (i.e., the presence) of the product of the reporter gene sequence. Finally, the expression and/or steroid binding-capacity of the hormone receptor protein (coded for by the receptor DNA sequence on the expression plasmid and produced in the transfected and cultured host cells), is measured by determining the activity of the reporter gene in the presence and absence of an antagonist. The antagonist activity of a compound may be determined in comparison to known antagonists of the glucocorticoid receptor and MR receptors (see, e.g., U.S. Pat. No. 5,696,127). Efficacy is then reported as the percent maximal response observed for each compound relative to a reference antagonist compound. A glucocorticoid receptor-specific antagonist is considered to exhibit at least a 100-fold, often 1000-fold or greater, activity towards the glucocorticoid receptor relative to the MR.

Glucocorticoid Receptor Antagonists as Pharmaceutical Compositions

The glucocorticoid receptor antagonists used in the methods of the invention can be administered by any means known in the art, e.g., parenterally, topically, orally, or by local administration, such as by aerosol or transdermally. The methods of the invention provide for prophylactic and/or therapeutic treatments. The glucocorticoid receptor antagonists as pharmaceutical formulations can be administered in a variety of unit dosage forms depending upon the condition or disease and the degree of postpartum psychosis, the general medical condition of each patient, the resulting preferred method of administration and the like. Details on techniques for formulation and administration are well described in the scientific and patent literature, see, e.g., the latest edition of Remington's Pharmaceutical Sciences, Maack Publishing Co, Easton Pa. (“Remington's”). Therapeutically effective amounts of glucocorticoid blockers suitable for practice of the method of the invention will typically range from about 0.5 to about 25 milligrams per kilogram (mg/kg). A person of ordinary skill in the art will be able without undue experimentation, having regard to that skill and this disclosure, to determine a therapeutically effective amount of a particular glucocorticoid blocker compound for practice of this invention. For example, a particular glucocorticoid blocker may be more effective at higher or lower doses. By evaluating a patient using the methods described herein, a skilled practitioner will be able to determine whether a patient is responding to treatment and will know how to adjust the dosage levels accordingly.

In general, glucocorticoid blocker compounds may be administered as pharmaceutical compositions by any method known in the art for administering therapeutic drugs. Compositions may take the form of tablets, pills, capsules, semisolids, powders, sustained release formulations, solutions, suspensions, elixirs, aerosols, or any other appropriate compositions; and comprise at least one compound of this invention in combination with at least one pharmaceutically acceptable excipient. Suitable excipients are well known to persons of ordinary skill in the art, and they, and the methods of formulating the compositions, may be found in such standard references as Alfonso A R: Remington's Pharmaceutical Sciences, 17th ed., Mack Publishing Company, Easton Pa., 1985. Suitable liquid carriers, especially for injectable solutions, include water, aqueous saline solution, aqueous dextrose solution, and glycols.

Aqueous suspensions of the invention contain a glucocorticoid receptor antagonist in admixture with excipients suitable for the manufacture of aqueous suspensions. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Oil suspensions can be formulated by suspending a glucocorticoid receptor antagonist in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

Glucocorticoid blocker pharmaceutical formulations can be prepared according to any method known to the art for the manufacture of pharmaceuticals. Such drugs can contain sweetening agents, flavoring agents, coloring agents and preserving agents. Any glucocorticoid blocker formulation can be admixtured with nontoxic pharmaceutically acceptable excipients which are suitable for manufacture.

Typically, glucocorticoid blocker compounds suitable for use in the practice of this invention will be administered orally. The amount of a compound of this invention in the composition may vary widely depending on the type of composition, size of a unit dosage, kind of excipients, and other factors well known to those of ordinary skill in the art. In general, the final composition may comprise from 0.000001 percent by weight (% w) to 10 % w of the glucocorticoid blocker compounds, preferably 0.00001% w to 1% w, with the remainder being the excipient or excipients. For example, the glucocorticoid receptor antagonist mifepristone is given orally in tablet form, with dosages in the range of between about 0.5 and 25 mg/kg, more preferably between about 0.75 mg/kg and 15 mg/kg, most preferably about 10 mg/kg.

Pharmaceutical formulations for oral administration can be formulated using pharmaceutically acceptable carriers well known in the art in dosages suitable for oral administration. Such carriers enable the pharmaceutical formulations to be formulated in unit dosage forms as tablets, pills, powder, dragees, capsules, liquids, lozenges, gels, syrups, slurries, suspensions, etc. suitable for ingestion by the patient. Pharmaceutical preparations for oral use can be obtained through combination of glucocorticoid blocker compounds with a solid excipient, optionally grinding a resulting mixture, and processing the mixture of granules, after adding suitable additional compounds, if desired, to obtain tablets or dragee cores. Suitable solid excipients are carbohydrate or protein fillers and include, but are not limited to sugars, including lactose, sucrose, mannitol, or sorbitol; starch from corn, wheat, rice, potato, or other plants; cellulose such as methyl cellulose, hydroxypropylmethyl-cellulose or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

The glucocorticoid receptor antagonists of this invention can also be administered in the form of suppositories for rectal administration of the drug. These formulations can be prepared by mixing the drug with a suitable non-irritating excipient which is solid at ordinary temperatures but liquid at the rectal temperatures and will therefore melt in the rectum to release the drug. Such materials are cocoa butter and polyethylene glycols.

The glucocorticoid receptor antagonists of this invention can also be administered by in intranasal, intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995).

The glucocorticoid receptor antagonists of the invention can be delivered by transdermally, by a topical route, formulated as applicator sticks, solutions, suspensions, emulsions, gels, creams, ointments, pastes, jellies, paints, powders, and aerosols.

The glucocorticoid receptor antagonists of the invention can also be delivered as microspheres for slow release in the body. For example, microspheres can be administered via intradermal injection of drug (e.g., mifepristone)-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

The glucocorticoid receptor antagonist pharmaceutical formulations of the invention can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5, that is combined with buffer prior to use.

In another embodiment, the glucocorticoid receptor antagonist formulations of the invention are useful for parenteral administration, such as intravenous (IV) administration. The formulations for administration will commonly comprise a solution of the glucocorticoid receptor antagonist (e.g., mifepristone) dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of glucocorticoid receptor antagonist in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the glucocorticoid receptor antagonist formulations of the invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the glucocorticoid receptor antagonist into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

After a pharmaceutical comprising a glucocorticoid receptor antagonist of the invention has been formulated in a acceptable carrier, it can be placed in an appropriate container and labeled for treatment of an indicated condition. For administration of glucocorticoid receptor antagonists, such labeling would include, e.g., instructions concerning the amount, frequency and method of administration. In one embodiment, the invention provides for a kit for treating postpartum psychosis in a subject which includes a glucocorticoid receptor antagonist and instructional material teaching the indications, dosage and schedule of administration of the glucocorticoid receptor antagonist.

Determining Dosing Regimens for Glucocorticoid Receptor Antagonists

The methods of this invention treat postpartum psychosis in a subject. The amount of glucocorticoid receptor antagonist adequate to accomplish this is defined as a “therapeutically effective dose”. The dosage schedule and amounts effective for this use, i.e., the “dosing regimen,” will depend upon a variety of factors, including the severity of the psychosis, the patient's physical status, age and the like. In calculating the dosage regimen for a patient, the mode of administration also is taken into consideration.

The dosage regimen also takes into consideration pharmacokinetics parameters well known in the art, i.e., the glucocorticoid receptor antagonists' rate of absorption, bioavailability, metabolism, clearance, and the like (see, e.g., Hidalgo-Aragones, J. Steroid Biochem. Mol. Biol. 58:611-617, 1996; Groning, Pharmazie 51:337-341, 1996; Fotherby, Contraception 54:59-69, 1996; Johnson, J. Pharm. Sci. 84:1144-1146, 1995; Rohatagi, Pharmazie 50:610-613, 1995; Brophy, Eur. J. Clin. Pharmacol. 24:103-108, 1983; the latest Remington's, supra). For example, in one study, less than 0.5% of the daily dose of mifepristone was excreted in the urine; the drug bound extensively to circulating albumin (see Kawai, supra, 1989). The state of the art allows the clinician to determine the dosage regimen for each individual patient, glucocorticoid receptor antagonist and disease or condition treated. As an illustrative example, the guidelines provided below for mifepristone can be used as guidance to determine the dosage regiment, i.e., dose schedule and dosage levels, of any glucocorticoid receptor antagonist administered when practicing the methods of the invention.

Single or multiple administrations of glucocorticoid receptor antagonist formulations can be administered depending on the dosage and frequency as required and tolerated by the patient. The formulations should provide a sufficient quantity of active agent, i.e., mifepristone, to effectively treat postpartum psychosis in a subject. For example, a typical preferred pharmaceutical formulation for oral administration of an antiglucocorticoid such as mifepristone or ORG 34517 would be about 5 to 15 mg/kg of body weight per patient per day, more preferably between about 8 to about 12 mg/kg of body weight per patient per day, most preferably 10 mg/kg of body weight per patient per day, although dosages of between about 0.5 to about 25 mg/kg of body weight per day may be used in the practice of the invention. Lower dosages can be used, particularly when the drug is administered to an anatomically secluded site, such as the cerebral spinal fluid (CSF) space, in contrast to administration orally, into the blood stream, into a body cavity or into a lumen of an organ. Substantially higher dosages can be used in topical administration. Actual methods for preparing parenterally administrable glucocorticoid receptor antagonist formulations will be known or apparent to those skilled in the art and are described in more detail in such publications as Remington's, supra. See also Nieman, In “Receptor Mediated Antisteroid Action,” Agarwal, et al., eds., De Gruyter, New York, 1987.

### EXAMPLES

The following examples are offered to illustrate, but not to limit the claimed invention.

### Example 1

Treating Postpartum Psychosis with Mifepristone

The following example illustrates the methods of the invention.

A 32-year old woman without prior history of psychotic illness is experiencing delusions and hallucinations two months after giving birth to her first child. The woman is diagnosed with postpartum psychosis (as described above) and is admitted to a psychiatric hospital. The woman's diagnosis of postpartum psychosis is confirmed by two psychiatrists and she is admitted for a nine day, closely observed hospital stay.

The woman is treated with a glucocorticoid receptor antagonist, mifepristone, administered in dosages of about 15 mg per kg once daily over a relatively short period. Thus, daily doses of mifepristone, in the range of 800 mg per day, over about a four day period will be used as an effective treatment for postpartum psychosis.

During the course of her hospital stay and treatment, the woman's progress will be monitored by application of the “Brief Psychiatric Rating Scale (BPRS)” test (Overall (1962) Psychol. Rep. 10:799). Results of the BPRS will be evaluated according to the seven-point “Likert Scale” from “not present” to “extremely severe”, thereby providing a quantitative score reflective of global pathology. Thus, the BPRS will provide the barometer of the woman's overall benefit from treatment using the methods of the invention. The BRPS Rating Scale will be given both before and after administration of mifepristone.

In conjunction with the BPRS test, the woman's diagnosis, categorization, or treatment success will be objectively assessed using tests such as that described by Wallach (1980) J. Gerontol. 35:371-375, or the Stroop Color and Word Test. However, any test known in the art would be equally effective for her evaluation.

The Brief Psychiatric Rating Scale (BPRS) (Overall, J. E. and Gorham, D. R. (1962) supra) will be carried out on days one, three, five, seven and nine. Other tests, such as the “Wallach Recognition Test”, will be given on days one, five and nine.

The woman will be given 800 milligrams of mifepristone once per day orally, over four days.

At the end of her hospital stay, the woman's Brief Psychiatric Rating Scale (BPRS) scores are expected to decline from about 40.5 to about 29.5. When the results of the Wallach Recognition Test are evaluated, the woman will show an amelioration of postpartum psychosis. Indeed, the number of distracting words misidentified as words actually presented in the test is expected to decline between 25% and 100% after treatment.

This example illustrates how doses of mifepristone, in the range of about 800 mg per day, given once daily over a relatively short period of time—about four days—are expected to produce an effective and safe treatment for postpartum psychosis.

It is understood that the examples and embodiments described herein are for illustrative purposes only and that various modifications or changes in light thereof will be suggested to persons skilled in the art and are to be included within the spirit and purview of this application and scope of the appended claims.

What is claimed is:

1. A method of ameliorating the symptoms of postpartum psychosis in a patient in need thereof, comprising administering an amount of a glucocorticoid receptor antagonist effective to ameliorate the symptoms of the postpartum psychosis, with the proviso that the first psychotic symptoms arise within nine months of childbirth, that the patient has never suffered any psychotic condition not triggered by childbirth, and that the patient did not suffer from psychosis prior to parturition.

2. The method of claim 1, wherein the first psychotic symptoms arise within eight weeks of childbirth.

3. The method of claim 1, wherein the glucocorticoid receptor antagonist comprises a steroidal skeleton with at least one phenyl-containing moiety in the 11-β position of the steroidal skeleton.

4. The method of claim 3, wherein the phenyl-containing moiety in the 11-β position of the steroidal skeleton is a dimethylaminophenyl moiety.

5. The method of claim 4, wherein the glucocorticoid receptor antagonist comprises mifepristone.

6. The method of claim 4, wherein the glucocorticoid receptor antagonist is selected from the group consisting of 11β-(4-dimethylaminoethoxyphenyl)-17α-propynyl-17β-hydroxy-4,9 estradien-3-one and 17β-hydroxy-17α-19-(4-methylphenyl)androsta-4,9(11)-dien-3-one.

7. The method of claim 1 wherein the glucocorticoid receptor antagonist is selected from the group consisting 4α(S)-Benzyl-2(R)-prop-1-ynyl-1,2,3,4,4α,9,10,10α(R)-octahydro-phenanthrene-2,7-diol and 4(S)-Benzyl-2(R)-chloroethynyl-1,2,3,4,4α,9,10,10α(R)-octahydro-phenanthrene-2,7-diol.

8. The method of claim 1, wherein the glucocorticoid receptor antagonist is (11β,17β)-11-(1,3-benzodioxol-5-yl)-17-hydroxy-17-(1-propynyl)estra-4,9-dien-3-one.

9. The method of claim 1, wherein the administration is once per day.

10. The method of claim 1, wherein the mode of administration is oral.

11. The method of claim 1, wherein the mode of administration is by a transdermal application, by a nebulized suspension, or by an aerosol spray.

12. A kit for treating postpartum psychosis in a human, the kit comprising:
(i) a specific glucocorticoid receptor antagonist; and,
(ii) an instructional material teaching the indications, dosage and schedule of administration of the glucocorticoid receptor antagonist to a patient with postpartum psychosis.

13. The kit of claim 12, wherein the glucocorticoid receptor antagonist is mifepristone.

14. The kit of claim 12, wherein the mifepristone is in tablet form.
